(19) United States
(12) Patent Application Publication
(10) Pub. No.: US 2007/0270598 A1
(43) Pub. Date: Nov. 22, 2007

(54) Process for one pot conversion of artemisinin into artelinic acid

(75) Inventors: Rajendra Singh Bhakuni, Lucknow (IN); Tarun Singh, Lucknow (IN); Rinku Singh, Lucknow (IN); Atul Prakash Kahol, Lucknow (IN); Suman Preet Singh Khanuja, Lucknow (IN)

Correspondence Address: SUGHRUE MION, PLLC, 2100 PENNSYLVANIA AVENUE, N.W., SUITE 800, WASHINGTON, DC, 20037, US

(73) Assignee: COUNCIL OF SCIENTIFIC & INDUSTRIAL RESEARCH, New Delhi (IN)

(21) Appl. No.: 11/716,045

(22) Filed: Mar. 9, 2007

(30) Foreign Application Priority Data
Mar. 10, 2006 (IN) 0670/DEL/2006

(51) Int. Cl. C07D 313/06

(52) U.S. Cl. 549/354

(57) ABSTRACT

The present invention relates to an improved process for one pot conversion of artemisinin into artelinic acid, which reduces the three step (three pot) conversion of artemisinin to artelinic acid in one step (one pot). The process of preparation of artelinic acid involves stirring of artemisinin with sodium borohydride, catalyst, polyhydroxy compound or chlorotrimethylsilane or amberlyst-15 resin and methyl p-(hydroxymethyl) benzoate, filtration of undissolved, unwanted reaction products and finally stirring of the filtrate with alcoholic or aqueous alkali hydroxide.

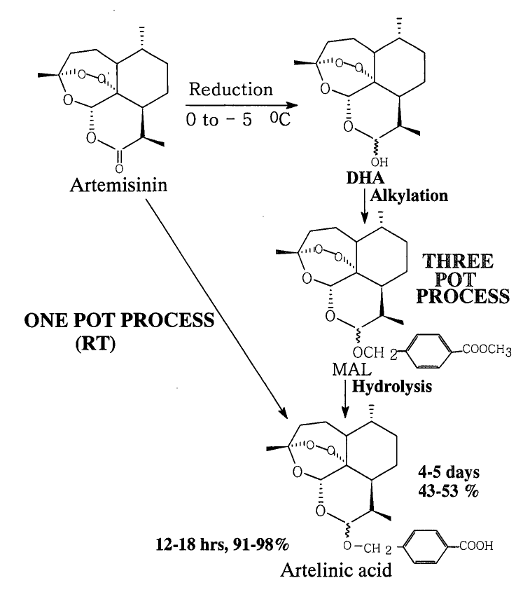

### FIELD OF INVENTION

The present invention relates to an improved process for one pot conversion of artemisinin into artelinic acid, which reduces the three step (three pots) conversion of artemisinin to artelinic acid in one step (one pot). Artelinic acid and sodium artelinate are customary names for p[(12-dihydroartemisininoxy)methyl] benzoate and sodium p-[12-dihydroartemisininoxy] methyl] benzoate, respectively.

### BACKGROUND OF THE INVENTION

Malaria is caused by protozoan parasites, notably Plasmodium falciparum. An approximately out of the 4 billion people suffering from malaria, 1-3 million, mostly children die every year worldwide. The range of drugs available for malaria is limited, and there are problems of drug resistance. Artemisinin and its derivatives: artemether and arteether (oil solubles), artelinate and artesunate (water sulables), a novel class of antimalarials derived from Artemisia annua are now proving their promising activity and are being used for the treatment of uncomplicated/severe complicated/cerebral and multi drug resistant malaria. The chemistry and the antiprotozoal action of these compounds is described in the following publications: Woerdenbag et al., Progress in the Research of Artemisinin Related Antimalarials: An Update, Pharm. World Sci. 16 (4), 169-180 (1994); Hien et al., Qinghaosu, The Lancet 341, 603-608. (1993); Butler et al., Artemisinin (Qinghaosu): A New Type of Antimalarial Drug, Chem. Soc. Reviews, 85-90(1992); Zaman et al., Some Aspects of the Chemistry and Biological Activity of Artemisinin and Related Antimalarials, Heterocycles 32 (8), 1593-1638 (1991); Woerdenbag et al., Artimisia annua L.:A Source of Novel Antimalarial Drugs, Pharm. Weekblad Sci. 12(5), 169-181 (1990); Klayman, D. L., Qinghaosu (Artemisinin): An Antimalarial Drug from China, Science 228, 1049-1055 (1985).

The water-insoluble artesunic acid and artelenic acid is customarily administered orally in the form of tablets or rectally in the form of suppositories, while the water-soluble artesunate and artelinate are administered intravenously.

Although sodium salt of artesunic acid, the half succinic acid ester derivative of DHA was clinically evaluated in China as water soluble drug but its utility was impaired by poor stability in aqueous solution due to the easy hydrolysis of its ester linkage.

A new water soluble derivative was needed not only to overcome the instability problem in the aqueous solution but also of having much longer plasma half life than artemether, arteether and artesunate. The sodium artelinate is considered the best candidate drug amongst the available water soluble analogues of this class for the treatment of multi drug resistant and cerebral malaria.

Lin et al., J. Med. Chem, 30, 2147-2150 (1987) prepared β-artelinic acid in three steps (three pots) (Chart-I) and and its sodium salts four steps (four pots), respectively. Artemisinin was converted into dihydroartemisinin (According to the method reported by Brossi et al) in the first step (1^{st} Pot); In the second step (2^{nd} pot) the dihydroartemisinin was stirred in ether with methyl-p(hydroxymethyl) benzoate in the presence of BF₃.Et₂O an acid catalyst for 24 hrs. The reaction mixture was washed with NaHCO₃ and H₂O, dried with Na₂SO₄ and evaporate to dryness to give oily product. The impure product was purified by column chromatography to yield methyl artelinate. In the third step (3^{rd} pot) methyl artelinate was hydrolysed with 2.5 NaOH/MeOH solution for two days at room temp and after acidic workup β artelinic acid was obtained in 55% yield in the step (43.5% with respect to artemisinin). In the last step β artelinic acid was converted into water soluble sodium β-artelinate.

Another three steps (three pots) (Chart-I) method for the preparation of α-artelinic acid was reported by Vishwakarma et al J. Nat. Product, 55, 114-1144(1992). In this method dihydroartemisinin was stirred with methyl-p(iodomethyl) benzoate in dry dichloromethane using freshly prepared Ag₂O for 5 hrs at room temperature and yielded only α-epimer, methyl α artelinate. Hydrolysis and subsequent preparation of sodium salt of the acid, methyl α artelinate was done according to the method reported by lin et al.

In another three pots method reported by Jain et al, U.S. Pat. No. 6,346,631, and Indian Patent No. 185198, for the preparation of the β artelinic acid, in the second pot methyl artelinate was prepared by stirring dihydroartemisinin with methyl-p(hydroxymethyl) benzoate in the presence of a different acid catalyst chlorotrimethylsilane in dry benzene for 2-6 hrs. The reaction mixture was washed with sodium acetate, dried with sodium sulphate and evaporated to dryness to yield oily product which upon column chromatography yielded pure methyl artelinate. This methodology thus, starting from artemisinin yielded β-artelinic acid (53%) (Chart-I) which was furthur converted into sodium artelinate.

In another method reported by Bhakuni et al, a novel one pot conversion of artemisinin into arteether, U.S. Pat. No. 6,750,356, 15 Jun. 2004 in which artemisinin was converted into arteether using a new catalyst, polyhydroxy compound (PHC) which catalyses the reduction of artemisinin into dihydroartemisinin at room temperature (20-30 degree C.).

Another method reported by Singh et al, Tet. Lett 43, 7235-7237(2002), artemisinin was converted into methyl artelinate in 49% yield in one step by stirring artemisinin first with sodium borohydride and catalyst, Amberlyst-15 in tetrahydrofuran followed by replacing tetrahydrofuran with dichloromethane and strring further the reaction mixture with methyl (p-hydroxy methyl) benzoate at room temperature in 18 hrs.

The above methods for the preparation of artelinic acid carry some disadvantages as compared to the present invention are:
(a) All the above methods except the last one (incomplete method) require at least three steps (three separate pots) to convert artemisinin into artelinic acid i.e 1^{st} Pot: Conversion of artemisinin into dihydroartemisinin, 2^{nd} Pot: Covnersion of dihydroartemisinin into methyl artelinate, 3^{rd} Pot: Hydrolysis of methyl artelinate into artelinic acid.
(b) Consumes more chemicals being less cost effective and more time consuming.
(c) Reduces the yield of intermediates dihydroartemisinin and methyl artelinate and thus increase the production cost of artelinic acid and its salt.
(d) Further, the use of benzene is not acceptable according to the health standard as it is a carcinogenic solvent.
(e) The last prior art by Singh et al, Tet. Lett 43, 7235-7237(2002) which describes conversion of artemisinin into methyl artelinate in one pot in 49% yield is an incomplete methodology to convert artemisinin into artelinic acid. Another disadvantage of this process is the use of two solvents tetrahydrofuran and dichloromethane in one pot. Thus, after completion of reduction of artemisinin into dihydroartemisinin the solvent, tetrahydrofuran was evaporated and replaced by second solvent dichloromethane makes the process expensive and time taking.
(f) All above processes uses column chromatography in the purification of methyl artelinate/artelinic acid which is an extra chemical consuming, product yield lowering and time taking step.

Our invention is able to overcome the shortcoming of the cited invention above being one pot conversion of artemisinin into artelinic acid around 98% yield.

References

Shaofeng et al., Sup. 3 H Labeling of QHS Derivatives, Bull. Chin. Materia Medica, 6 (4), 25-27 (1981).
   Li et al., Synthesis of Ethers, Carboxylic Esters and Carbonates of Dihydroarthemisinin, Acta Pharm. Sin., 16(6), 429-439 (1981).
Ying et al. Synthesis of Some Carboxylic Esters and Carbonates of Dihydroartemisinin by Using 4-(N,N-Dimethylamino) pyridine as an Active Acylation Catalyst, Acta Chim,. Sinica 40 (6), 557-561 (1982)
Li et al., Chemical Abstracts, Vol. 98, No. 1, 3 Jan. 1983, abstract No. 4420 h.
Klayman, D. L., Qinghaosu (Artemisinin): An Antimalarial Drug from China, Science 228, 1049-1055 (1985).
Lin et al., Chemical Abstracts, Vol. 107, No. 19, 9 Nov. 1987, abstract No. 168241f.
Lin et al., Antimalarial Activity of New Water Soluble Dihydroartemisinin Derivatives, J. Med. Chem., Vol. 30, No. 11, pp 2147-2150. (1987).
Brossi et al., Arteether, A New Antimalarial Drug: Synthesis and Antimalarial Properties, J. Med. Chem. 31, 645-650 (1988).
El-Feraly et al., A New Method for the Preparetion of Arteether and Its C-9 Epimer, J. Nat. Prod., 55, 878-883(1992).
Chau et al. Chemical Abstracts, vol. 119, No. 23, 6 Dec. 1993, abstract No. 249761a,
Bhakuni et al, An Improved Method for the Preparation of Arteether from Dihydroartemisinin, Indian J. Chem. 34 B, 529-30(1995)
Lin et al., Antimalarial Activity of New Dihydroartemisinin Derivative. 6. α-Alkylbenzylic Eehers, J. Med. Chem., 38, 764-770(1995)
Bhakuni et al, A New Method for the Preparation of Arteether, an Antimalarial Drug. Indian Patent No. 80500, 8 Jan. (1999)
Shrimali et al., An Improved Process for the Preparation of Sodim Artelinate, a Water Soluble Potent Antimalarial Compound, Indian Patent No. 185198, 7 Jul. (2001)
Bhakuni et al., Single Pot Conversion of Artemisinin into Artemether, U.S. Pat. No. 6,683,193, 27 Jan. 2004
Bhakuni et al, A One pot Conversion of Artemisinin into Artesunic acid, U.S. Pat. No. 6,677,462, 13 Jan. 2004.
Ognyanov et al, Process for the Preparation of Dihydroartemisinin Hemisuccinate, U.S. Pat. No. 5,654,446, Aug. 5(1997).

### OBJECTIVES OF THE INVENTION

The main objective of the present invention is to provide a process for one pot conversion of artemisinin into artelinic acid.

The another objective of the present invention is to provide a process where, the yield of the product pure artelinic acid is up to 98%.

Another objective of the present invention is the development of one pot conversion of artemisinin into β artelenic acid which possesses reduction of artemisinin into dihydroartemisinin followed by alkylation of dihydroartemisinin to methyl artelinate, filtration of unwanted, undissolve reaction products and hydrolysis of methyl artelinate into β-artelinic acid in the filtrate in one pot at room temperature (20-30 degree C.).

Another objective of the present invention is the purification of impure product to pure artelinic acid without the use of column chromatography.

### SUMMARY OF THE INVENTION

The present invention relates to an improved process for one pot conversion of artemisinin into artelinic acid, which reduces the three step (three pot) conversion of artemisinin to artelinic acid in one step (one pot). Artelinic acid and sodium artelinate are customary names for p[(12-dihydroartemisininoxy)methyl] benzoate and sodium p-[12-dihydroartemisininoxy] methyl] benzoate, respectively.

The process of preparation of artelinic acid involves stirring of artemisinin with sodium borohydride ,catalyst, polyhydroxy compound (PHC—dextrose/galactose/phloroglucinol)—chlorotrimethylsilane or amberlyst-15 resin and methyl p-(hydroxymethyl) benzoate, filtration of undissolve, unwanted reaction products and finally stirring of the filtrate with alcoholic or aqueous alkali hydroxide.

It involves three step reactions: 1^{st}—reduction of artemisinin into dihydroartemisinin (DHA), 2^{nd}—alkylation of dihydroartemisinin into methyl artelinate (MAL), 3^{rd}—after filtration hydrolysis of methyl artelenate into artelinic acid in the filtrate were carried out in one pot (Chart-I) thus, avoids the unwanted process of isolation of dihydroartemisinin and methyl artelinate (MAL) which saves losses of the intermediates dihydroartemisinin and methyl artelinate, chemicals, labour and time and removal of unwanted, undissolve reaction products by filtration improves hydrolysis of methyl artelinate to produce artelinic acid in very high yield which does not require column chromatography in its purification.

### DETAILED DESCRIPTION OF THE INVENTION

Accordingly the present invention provides a one pot process for the preparation of β artelinic acid from artemisinin, which comprises:
a. dissolving artemisinin in a solvent selected from 1,4-dioxan, tetrahydrofuran at a temperature ranging between 20-35 degree C.,
b. adding a catalyst selected from cation exchange resin (Amberlyst 15), a polyhydroxy compound selected from a group consisting of dextrose, galactose and phloroglucinol to above solution of artemisinin,
c. adding a reducing agent and stirring the reaction mixture for about 2.5-8.0 hrs at a temperature ranging between 20-30 degree C. to obtain dihydroartemisinin,
d. adding methyl p(hydroxymethyl)benzoate and chlorotrimethylsilane (CTMS) to the above reaction mixture to obtain methyl artelinate,
e. filtering the reaction mixture to remove undissolve, unwanted reaction products,
f. adding a base such as an alkali hydroxide solution selected from group consisting of 5-15% KOH/MeOH or EtOH or H₂O to the above said filtrate obtained in step (f) and stirring the reaction mixture further for a period ranging 2-2.5 hrs at a temperature ranging between 30-35° C.,
g. adding cooled water in the reaction mixture and adjusting pH in the range of 5-7 with acetic acid followed by extraction with ethyl acetate,
h. washing the combined ethyl acetate extract with water;
i. drying the extract over dehydrating agent followed by concentration,
j. purifying the β-artelinic acid by chromatography if required or by its recrystallization with ethyl acetate-hexane to obtain pure product β-artelinic acid in very higher yield.

In the process of present invention artemisinin, polyhydroxy compound (PHC)—chlorotrimethylsilane or Amberlyst-15, sodium borohydride and methyl p-(hydroxymethyl) benzoate were stirred in dioxane/tetrahydrofuran at room temperature (20-30 degree C.) for about 2.5-7.0 hrs. After completion of the reduction and alkylation reaction, the undissolve, unwanted reaction products was filtered and in the filtrate 5-15% KOH/MeOH or EtOH/H₂O was added slowly at room temperature to carry out the hydrolysis of methyl artelinate. The reaction mixture was stirred further for about 2-9 hrs. After completion of the hydrolysis cooled water was added. The reaction mixture was neutralized with 5-10% acetic acid and extracted with ethyl acetate: (2-3 times). The combined extract was washed with water, dried over anhydrous sodium sulphate and removal of the solvent furnished impure artelinic acid which was recrystallized in ethyl acetate-hexane to furnished pure artelinic acid, single on thin layer chromatography (TLC) in 91.2-98% w/w yield.

In an embodiment of the present invention a process wherein three reactions, 1^{st}—reduction of artemisinin into dihydroartemisinin, 2^{nd}—alkylation of dihydroartemisinin into methyl artelinate and 3^{rd}—after filtration of unwanted and undissolve reaction products hydrolysis of methyl artelinate into β-artelinic acid in the filtrate are carried out in single pot (Chart-I).

In an embodiment of the present invention a process wherein the process of isolation of dihydroartemisinin and methyl artelinate is not required, saves chemicals, labour, time and losses of dihydroartemisinin and methyl artelinate in isolating them.

In an embodiment of the present invention a process wherein the process of filtration of undissolve and unwanted reaction products improves hydrolysis of methyl artelinate to produce artelicic acid in very high yield which does not require column chromatography in its purification.

In an embodiment of the present invention a process wherein the undissolve and unwanted reaction products were not filtered purification of artelinic acid required column chromatography which has lowered the yield (76-85%) of the product and the process consumed extra chemicals, solvents and time.

In an embodiment of the present invention a process wherein conversion of artemisinin into impure artelinic acid takes place in about 5-9 hrs and is a less time consuming method.

In another embodiment of the present invention a process wherein the conversion of artemisinin into artelinic acid is carried out at room temperature (20-30 degree C.) and thereby avoiding the use of cooling unit.

In yet another embodiment of the present invention a process wherein the same solvent 1,4-dioxan/tetrahydrofuran is used in reduction, alkylation and hydrolysis steps and is cost effective.

In yet another embodiment of the present invention a process wherein artemisinin and sodium brohydride are used in 1:2.0-3.0 w/w ratio.

Further in an embodiment a process as claimed in claims 1, 2, 4, 5 and 6 wherein artemisinin and methyl p(hydroxymethyl) benzoate are used in 1:2.0-3.0 w/w ratio.

Further in an embodiment a process as claimed in claim 1 wherein, artemisinin and chlorotrimethylsilane are used in ratio ranging between 1:1-4 w/v.

In an embodiment of the present invention a process wherein artemisinin and cation exchange resin are used in 1:10.0 w/w.

In an embodiment of the present invention a process wherein catalyst cation exchange resin(Amberlyst-15)/PHC, used to carry out the reduction and alkylation at room temperature can be recovered, regenerated and reused.

In an embodiment of the present invention a process wherein hydrolysis is carried out with 5-15% KOH/MeOH or EtOH/H₂O at room temperature.

In an embodiment of the present invention a process wherein after completion of the reaction the extraction of artelinic acid from aqueous reaction mixture is carried out with ethyl acetate.

In an embodiment of the present invention a process wherein the purification of impure artelenic acid is carried by simple crystallization with mixture of ethyl acetate-hexane to yield pure β artelinic acid in very high yield, 91.2-98% w/w as compared to 43.5 to 53% yields obtained in prior arts.

In an embodiment of the present invention a process wherein the purification process of impure artelenic acid into pure product omits column chromatography step which is an extra chemical consuming, product yield lowering and time consuming process.

In an embodiment of the present invention a process wherein the conversion of artemisinin into pure β artelinic acid in very high yield takes 12-18 hrs is very less time consuming process as compared to prior arts (4-5 days).

In an embodiment of the present invention a process wherein the improved method overcomes the disadvantages of previously known processes and is suitable for the preparation of β-artelinic acid/sodium β-artelinate in large scale.

Process of the Present Follows Two Methodologies

1. First Methodology: Use of Column Chromatography

After completion of all three reactions, reduction, alkylation and hydrolysis followed by usual workup provides crude artelenic acid in about double of artemisinin which require column chromatography to produce lower yield of pure product. Though it is better than all the prior art processes but the drawback of this process is the unwanted and undissolved reaction products obtained in the reduction and alkylation processes which lead to get much complicated mixture of artelinic acid and require column chromatography to produce pure product.
2. Second Methodology: Filtration and Removal of Column Chromatography

Removal of unwanted and undissolved reaction products after reduction and akylation processes improve the hydrolysis of MAL in the filtrate and produce cleaner product which upon usual work up and recrystallization produce pure artelinic acid in very high yield (91 to 98%).

Therefore, the novel process steps in the second methodology of filtration of unwanted reaction products and removal of column chromatography thereby leading to very high yield of pure product is the novelty of our present invention and are non-obvious.

The following examples are given by the way of illustration and therefore should not be construed to limit the scope of the invention:

### EXAMPLES—USING FILTRATION STEP

### Example 1

Artemisinin (1.0 g), polyhydroxy compound (dextrose, 5.0 g), sodium borohydride (2.0 g) and methyl p-(hydroxymethyl) benzoate (2.1 g) and chlorotrimethylsilane (CTMS) (1.0 ml) were stirred in 1,4-dioxan (40 ml) at room temperature at 25° C. for about 7 hrs. It was filtered. The filterate was further stirred with 10% KOH/H₂O (75 ml) for about 2 hrs. The reaction mixture was neutralized with 5% CH₃COOH, extracted with ethyl acetate (2×60 ml). The ethyl acetate extract was dried over anhydrous sodium sulphate and evaporation of solvent yielded impure artelinic acid (1.25 gm) which was recrystallized in ethyl acetate-hexane to yield pure artelinic acid in 98% yield (980 mg). After thorough drying the pure artelinic acid, m.p 142-415° C. was characterized by spectral analysis.

### Example 2

Artemisinin (50 mg), PHC (dextrose, 250 mg), sodium borohydride (100 mg) and methyl p-(hydroxymethyl) benzoate (100 mg) and chlorotrimethylsilane (CTMS) (0.1 ml) were stirred in tetrahydrofuran (3 ml) at room temperature at 20° C. for about 8.0 hrs. After completion of the reduction and alkylation reaction it was filtered. In the filtrate, 10% KOH/MeOH (15 ml) was added slowly at room temperature and the reaction mixture was stirred further for 3.5 hours at room temp. After usual work up and purification through prep TLC, yielded 16 mg (32%) artelinic acid.

### Example 3

Artemisinin (50 mg), phloroglucinol (300 mg), sodium borohydride (150 mg) and methyl p-(hydroxymethyl) benzoate (150 mg) and chlorotrimethylsilane (CTMS) (0.05 ml) were stirred in tetrahydrofuran (4 ml) at room temperature at 25° C. for about 8.0 hrs. The reaction mixture was filtered. In the filtrate 10% KOH/MeOH (15 ml) was added slowly at room temperature and the reaction mixture was stirred further for 3 hours at room temp. After usual work up and purification through prep TLC yielded 11 mg (22%) pure artelinic acid.

### Example 4

Artemisinin (50 mg), PHC (galactose, 300 mg), sodium borohydride (150 mg) and methyl p-(hydroxymethyl) benzoate (150 mg) and chlorotrimethylsilane (CTMS) (0.2 ml) were stirred in dioxane (3 ml) at room temperature at 23° C. for about 8.0 hrs. The reaction mixture was filtered. In the filtrate, 5% KOH/EtOH (15 ml) was added slowly at room temperature and it was stirred further for 7 hours at room temp. After usual work up and purification through prep TLC, yielded 9 mg (18%) artelinic acid.

### Example 5

Artemisinin (500 mg), cation exchange resin (5.0 g), sodium borohydride (1.0 g) and methyl p-(hydroxymethyl) benzoate (1.0 g) were stirred in 1,4-dioxan (10 ml) at room temperature at 25° C. for about 2.5 hours. After completion of the reduction and alkylation reaction (Checked by TLC) it was filtered. In the filtrate, 15% KOH/ EtOH (15 ml) was added slowly at room temperature and the reaction mixture was stirred further for about 2 hrs at room temperature. Added cooled water (50 ml) in the reaction mixture and the pH of the aqueous reaction mixture was adjusted about 6.0 with dilute acitic acid and extracted with ethyl acetate (3×25 ml). The combined ethyl acetate extract was dried over anhydrous sodium sulphate and evaporation of the solvent yielded 0.515 g of impure artelinic acid which upon recrystallization with mixture of ethyl acetate-hexane furnished pure artelinic acid (0.456 g) in 91.2% w/w yield.

### Example 6

Artemisinin (500 mg), cation exchange resin (5.0 g), sodium borohydride (1.0 g) and methyl p-(hydroxymethyl) benzoate (1.0 g) were stirred in tetrahydrofuran (10 ml) at room temperature (27° C.) for about 2.0 hrs. After completion of the reduction and alkylation reaction, it was filtered. In the filtrate 10% KOH/MeOH (15 ml) was added slowly at room temperature and the reaction mixture was stirred further for 4 hrs at room temp. After usual work up and purification through recrystallization yielded 235 mg (47%) pure artelinic acid.

### EXAMPLES—USING COLUMN CHROMATOGRAPHY

### Example 7

Artemisinin (500 mg), PHC (dextrose, 3.0 g), sodium borohydride (1.0 g) and methyl p-(hydroxymethyl) benzoate (1.0 g) and chlorotrimethylsilane (CTMS) (0.5 ml) were stirred in 1,4-dioxan (15 ml) at room temperature (20° C.) for about 6.0 hrs. After completion of the reduction and alkylation reaction (Checked by TLC), 15% KOH/MeOH (15 ml) was added slowly at room temperature and the reaction mixture was stirred further for 2 hours at room temp. Added cooled water (50 ml) in the reaction mixture and the pH of the aqueous reaction mixture was adjusted about 6.0 with dilute acetic acid and extracted with 50% ethyl acetate in hexane (3×25 ml). The combined extract was washed with water (50 ml). The ethyl acetate n-hexane extract was dried over anhydrous sodium sulphate and evaporation of the solvent yielded 935 mg of crude artelinic acid which upon purification over silica gel column (1:5 ratio) with 15-25% ethyl acetate in hexane, furnished pure artelinic acid (380 mg) in 76% w/w yield.

### Example 8

Artemisinin (500 mg), cation exchange resin (5.0 g), sodium borohydride (1.0 g) and methyl p-(hydroxymethyl) benzoate (1.0 g) were stirred in 1,4-dioxan (10 ml) at room temperature (27° C.) (20-30 degree C.) for about 2.5 hours. After completion of the reduction and alkylation reaction (Checked by TLC) 15% KOH/EtOH (15 ml) was added slowly at room temperature and the reaction mixture was stirred further for 2-2.5 hours at room temperature. Added cooled water (50 ml) in the reaction mixture and the pH of the aqueous reaction mixture was adjusted about 6.0 with dilute acetic acid and extracted with 50% ethyl acetate in hexane (3×25 ml). The combined extract was washed with water (50 ml). The ethyl acetate n-hexane extract was dried over anhydrous Sodium sulphate and evaporation of the solvent yielded 900 mg of crude artelinic acid which upon purification over silica gel column (1:5 ratio) with 15-25% ethyl acetate in hexane, furnished pure artelinic acid in 82% w/w yield. After drying the pure artelinic acid, m.p 142-415° C. was characterized by spectral analysis.

### Example 9

Artemisinin (500 mg), cation exchange resin (5.0 g), sodium borohydride (1.5 g) and methyl p-(hydroxymethyl) benzoate (750 mg) were stirred in 1,4-dioxan (10 ml) at room temperature (30° C.) for about 3.0 hrs. After completion of the reduction and alkylation reaction (Checked by TLC ), 5% KOH/MeOH (15 ml) was added slowly at room temperature and the reaction mixture was stirred further for 24 hours at room temp. After usual work up and purification through column chromatography (1:4 ratio) yielded 150 mg (30%) pure artelinic acid.

### Example 10

Artemisinin (500 mg), cation exchange resin (5.0 g), sodium borohydride (1.0 g) and methyl p-(hydroxymethyl) benzoate (1.0 g) were stirred in tetrahydrofuran (10 ml) at room temperature (23° C.) (20-30 degree C.) for about 2.0 hrs. After completion of the reduction and alkylation reaction (Checked by TLC), 5% KOH/MeOH (15 ml) was added slowly at room temperature and the reaction mixture was stirred further for 24 hours at room temp. After usual work up and purification through column chromatography (1:4 ratio) yielded 100 mg (20%) pure artelinic acid.

### Example 11

Artemisinin (50 mg), PHC (100 mg), sodium borohydride (1.0 g) and methyl p-(hydroxymethyl) benzoate (1.0 g) were stirred in tetrahydrofuran (3 ml) at room temperature (30° C.) for about 4.0 hrs. 5% KOH/MeOH (15 ml) was added slowly at room temperature and the reaction mixture was stirred further for 24 hours at room temp. After usual work up and purification through prep TLC, yielded 4 mg (8%) artelinic acid.

### Example 12

Artemisinin (2 g), cation exchange resin (20.0 g), sodium borohydride (5.0 g) and methyl p-(hydroxymethyl) benzoate (4.0 g) were stirred in 1,4-dioxan (60 ml) at room temperature (23° C.) for about 3.0 hrs. After completion of the reduction and alkylation reaction (Checked by TLC) 15% KOH/EtOH(40 ml) was added slowly at room temperature and the reaction mixture was stirred further for 2-2.5 hrs at room temperature. Added cooled water (50 ml) in the reaction mixture and the pH of the aqueous reaction mixture was adjusted about 6.0 with dilute acetic acid and extracted with 50% ethyl acetate in hexane (3×50 ml). The combined extract was washed with water (500 ml). The ethyl acetate n-hexane extract was dried over anhydrous sodium sulphate and evaporation of the solvent yielded 3.96 g of crude artelinic acid which upon purification over silica gel column (1:5 ratio) with 15-25% ethyl acetate in hexane, furnished pure artelinic acid in 85% w/w yield.

Advantages

1. The three step reactions: 1^{st}—reduction of artemisinin into dihydroartemisinin 2^{nd}—alkylation of dihydroartemisinin into methyl artelinate 3^{rd}—hydrolysis of methyl artelenate into artelinic acid was carried out in one pot (Chart-I) thus, avoids the unwanted process of isolation of dihydroartemisinin and methyl artelinate which saves losses of the intermediates dihydroartemisinin and methyl artelinate, chemicals, labour and time.
   1. Conversion of artemisinin into impure artelinic acid in one pot takes place in about (5-9 hrs) and is a less time consuming method as compared to previously reported three pot conversion of artemisinin into dihydroartemisinin in first pot followed by isolation of dihydroartemisinin and its alkylation into methyl artelinate in the second pot, isolation of it and hydrolysis of methyl artelinate into artelinic acid in the third pot is a long time consuming process.
   2. After completion of alkylation reaction, the process of filtration of undissolve, unwanted reaction products improves hydrolysis of methyl artelinate to produce artelicic acid in very high yield.
   3. The process of purification of impure artelenic acid into pure product omits column chromatography step which is an extra chemical consuming, product yield lowering and time consuming process.
   4. The conversion of artemisinin into impure artelenic acid followed by workup and purification to yield pure product in very high yield takes 12-18 hrs as compared to previously reported methods (4-5 days) and thus the improved process is very less time consuming.
   5. The conversion of artemisinin into artelinic acid in one pot is carried out at room temperature (20-30 degree C.) and thereby avoids use of cooling unit as used in the conversion of artemisinin into dihydroartemesinin in the 1^{st} step in the prior arts.
   6. A process wherein the same solvent 1,4-dioxan/tetrahydrofuran is used in reduction, alkylation and hydrolysis steps and is cost effective.
   7. The catalysts, cation exchange resin used to carry out the reduction of artemisinin into dihydroartemisinin at room temperature (20-30 degree C.) can be regenerated and reused.
   8. The improved process provides increased yield of pure β-artelinic acid in 91.2-98% w/w as compared to 43.5 to 53% yields obtained in prior art processes.
   9. Thus, this improved method which avoids the disadvantages of previously known proceses is suitable for the preparation of β artelenic acid/Sodium artelinate in large scale.
   10. A process wherein after completion of the reaction the extraction of artelinic acid from aqueous reaction mixture is carried out with ethyl acetate is cost effective.
   11. A process as claimed in claim 1 wherein, after completion of alkylation reaction, the process of filtration of undissolve, unwanted reaction products improves hydrolysis of methyl artelinate to produce artelicic acid in very high yield.
   12. A process as claimed in claim 1 wherein, the process of purification of impure artelenic acid into pure product omits column chromatography step which is an extra chemical consuming, product yield lowering and time consuming process.

The improved method overcomes the disadvantages of previously known processes and is suitable for the preparation of β-artelinic acid/sodium β-artelinate in large scale.

We claim:

1. A one pot process for the preparation of β-artelinic acid from artemisinin, which comprises:
a. dissolving artemisinin in a solvent selected from 1,4-dioxan, tetrahydrofuran at a temperature ranging between 20-35 degree C.,
b. adding a catalyst selected from cation exchange resin (Amberlyst 15), a polyhydroxy compound selected from a group consisting of dextrose, galactose and phloroglucinol to above solution of artemisinin,
c. adding a reducing agent and stirring the reaction mixture for about 2.5-8.0 hrs at a temperature ranging between 20-30 degree C. to obtain dihydroartemisinin,
d. adding methyl p(hydroxymethyl)benzoate and chlorotrimethylsilane (CTMS) to the above reaction mixture to obtain methyl artelinate,
e. filtering the reaction mixture to remove undissolved, unwanted reaction products,
f. adding a base such as an alkali hydroxide solution selected from group consisting of 5-15% KOH/MeOH or EtOH or H₂O to the above said filtrate obtained in step (f) and stirring the reaction mixture further for a period ranging 2-2.5 hrs at a temperature ranging between 30-35° C.,
g. adding cooled water in the reaction mixture and adjusting pH in the range of 5-7 with acetic acid followed by extraction with ethyl acetate,
h. washing the combined ethyl acetate extract with water;
i. drying the extract over dehydrating agent followed by concentration,
j. purifying the β-artelinic acid by chromatography, if required, or by its recrystallization with ethyl acetate-hexane to obtain pure product β-artelinic acid in very higher yield.

2. A process as claimed in claim 1 wherein, the three steps to convert artemisinin to β-artelinic acid, 1^{st} reduction of artemisinin into dihydroartemisinin, 2^{nd}-alkylation of dihydroartemisinin into methyl artelinate and 3^{rd}—hydrolysis of methyl artelinate into β-artelinic acid are carried out in a single pot (Chart-I).

3. A process as claimed in claim 1 wherein, the reducing agent used is selected from the group consisting of sodium borohydride, lithium aluminium hydride, lithium tritertbutoxy aluminium gydride, lithium trimethoxy aluminium hydride, sodium trimethoxy borohydride, sodium bis-2-methoxy, ethoxy aluminium hydride and a mixture of lithium or sodium in alcohol or liquid ammonia, preferably sodium borohydride.

4. A process as claimed in claim 1 wherein, the alkali used for hydrolysis of methyl artelinate is selected from group consisting of KOH, NaOH, preferably 5-15% KOH/MeOH or EtOH or H₂O.

5. A process as claimed in claims 1 wherein, silylated liquid acid catalyst is chlorotrimethylsilane.

6. A process as claimed in claims 1 wherein, artemisinin and chlorotrimethylsilane are used in ratio ranging between 1:1-4 w/v.

7. A process as claimed in claims 1 wherein, artemisinin and sodium brohydride is used in ratio ranging between 1:2.0-3.0 w/w.

8. A process as claimed in claims 1 wherein, artemisinin and methyl p(hydroxymethyl) benzoate are used in ratio ranging between 1:2.0-3.0 w/w.

9. A process as claimed in claims 1 wherein, artemisinin and cation exchange resin are used in ratio ranging between 1:0.0 w/w.

10. A process as claimed in claims, 1 wherein, the catalyst cation exchange resin (Amberlyst-15) used to carry out the reduction and alkylation at room temperature can be recovered, regenerated and reused.

11. A process as claimed in claims, 1 wherein, hydrolysis is carried out using alcoholic or aqueous alkali selected from 5-15% KOH/MeOH or EtOH or H₂O at room temperature (30-35° C.).

13. A process as claimed in claims 1 wherein, the conversion of artemisinin into impure artelenic acid is followed by workup and purification to yield pure product in very high yield takes 12-18 hrs as compared to previously reported methods (4-5 days) and the improved process being very less time consuming.

14. A process as claimed in claims 1 wherein, the purification of artelenic acid is carried out by recrystallization with ethyl acetate-n-hexane mixture to yield pure product, β artelinic acid in higher yield, 91.2-98% w/w as compared to 43.5 to 53% yields obtained in prior arts.

15. A one pot process for the preparation of β artelinic acid from artemisinin substantially as herein described with reference to the examples and drawing accompanying the specification.
